(19) United States
(12) Patent Application Publication
(10) Pub. No.: US 2017/0354474 A1
(43) Pub. Date: Dec. 14, 2017

(54) TEE PROBE CONTAINER

(71) Applicant: AMT Group Bahamas Ltd., Nassau (BS)

(72) Inventor: Gregory John Dobbyn, Creedmoor, NC (US)

(21) Appl. No.: 15/617,803

(22) Filed: Jun. 8, 2017

Related U.S. Application Data
(60) Provisional application No. 62/347,838, filed on Jun. 9, 2016.

(51) Int. Cl. A61B 50/33; A61B 8/12; B65D 77/00; B65B 7/28; A61B 8/00; A61B 50/00

(52) U.S. Cl. CPC A61B 50/33; B65B 7/28; A61B 8/4422; A61B 8/12; B65D 77/003; A61B 2050/0084

(57) ABSTRACT

The present invention is directed to a TEE Probe container configured for receiving a TEE Probe, the container comprising a first container surface and an opposing second container surface. In some instances, the container further comprises one or more depressions positioned on the first surface configured for receiving the TEE probe. The TEE probe container further comprises a planar lid configured to removably enclose the first container surface of the container.

### CROSS-REFERENCE TO PRIORITY APPLICATION

This application claims the benefit of U.S. Provisional Patent Application Ser. No. 62/347,838 entitled “TEE Probe Container” filed Jun. 9, 2016, which is hereby incorporated by reference in its entirety.

### FIELD OF THE INVENTION

This invention relates to, in general, operation and maintenance of transesophageal echocardiogram (TEE) probes, endoscopes, medical/surgical probes and other medical instruments.

### BACKGROUND

Echocardiogram, often referred to as a cardiac echo, or simply an echo, is a sonogram of the heart. This imaging technique takes on a number of forms, one of which is the TEE, as discussed above. TEE imaging offers some advantages over other cardiac imaging techniques as it allows the ultrasound probe to be placed within a patient's chest cavity in close proximity to the patient's heart. Other techniques, such as the transthoracic echocardiogram, or TTE, are more common, but utilize an ultrasound device designed to image the heart from outside the chest cavity. These devices use ultrasound waves that must traverse skin, fat, ribs, and lungs before reflecting off the heart and back to the probe. Since the heart rests directly upon the esophagus, the TEE transducer probe is situated in a better position to provide a clear sonogram image, as the ultrasound beam must only travel millimeters during the TEE probe operation. The TEE comparatively reduces attenuation of the ultrasound signal, which ultimately enhances image quality. A TEE probe typically contains an elongated probe or an elongated probe shaft with a transducer device at its tip that is inserted into the patient's esophagus, thereby placing the ultrasound device in close proximity to the heart and allowing the heart to be imaged in detail.

There is a need for systems and methods to streamline the sanitary use and practical utilization of the TEE probe such that the TEE probe may be easily and economically transported, sanitized, and reused.

### BRIEF SUMMARY

The following presents a simplified summary of one or more embodiments of the invention in order to provide a basic understanding of such embodiments. This summary is not an extensive overview of all contemplated embodiments, and is intended to neither identify key or critical elements of all embodiments, nor delineate the scope of any or all embodiments. Its sole purpose is to present some concepts of one or more embodiments in a simplified form as a prelude to the more detailed description that is presented later.

Embodiments of the invention are directed to the design and methods for using a TEE probe container. The invention generally embodies a container designed to receive a TEE probe and its various components, as well as a planar lid configured to removably enclose the container surface. Furthermore, the container surface might contain lid fasteners in some embodiments that may be arranged in any number of known combinations that are possible to provide complimentary designs for the position of the fasteners complimentary to the design of a reversible, planar lid. As such, in some embodiments, the TEE Probe container comprises: a container configured for receiving a TEE Probe, the container comprising a first container surface and an opposing second container surface; a lid configured to removably enclose the first container surface of the container; wherein the container comprises: a first depression positioned on the first container surface configured for receiving the TEE probe; a second depression positioned on the first container surface configured for receiving a sponge; a third depression positioned on the first container surface configured for receiving a mouthpiece; and a fourth depression positioned on the first container surface configured for receiving a flexible probe cover that is configured to at least partially cover the TEE probe (for example, the flexible probe cover/sleeve is configured to cover the TEE probe shaft). In alternative embodiments, the TEE Probe container comprises: a container configured for receiving a TEE Probe, the container comprising a first container surface and an opposing second container surface; a planar lid configured to removably enclose the first container surface of the container; wherein the container comprises: a first depression positioned of the first container surface configured for receiving the TEE probe; a second depression positioned of the first container surface configured for receiving a sponge; and a third depression positioned of the first container surface configured for receiving a mouthpiece.

In some embodiments, or in combination with the previous embodiment, the container further comprises a guard positioned on and extending along a perimeter of the first container surface.

In some embodiments, or in combination with any of the previous embodiments, the first depression, the second depression, the third depression, and the fourth depression are discrete. In alternative embodiments, or in combination with any of the previous embodiments, the first depression, the second depression and the third depression are discrete.

In some embodiments, or in combination with any of the previous embodiments, the TEE Probe Container further comprises a flexible probe cover or the flexible sleeve configured to encase at least a portion of the TEE probe, for example the TEE probe shaft. In some embodiments, or in combination with any of the previous embodiments, the flexible probe cover is configured to encase the pre-patient use TEE Probe. In some embodiments, or in combination with any of the previous embodiments, the TEE probe container of claim 1, the flexible probe cover is configured to encase the post-patient use TEE Probe.

In some embodiments, or in combination with any of the previous embodiments, the TEE Probe Container further comprises a flexible container bag configured to encase the container. In some embodiments, or in combination with any of the previous embodiments, the TEE Probe Container further comprises a flexible lid bag configured to encase the lid.

In some embodiments, or in combination with any of the previous embodiments, the lid is operatively coupled to the container through one or more fastening members, wherein the fastening members comprise mating protrusions and apertures, groove elements and tab projections, groove elements and a portions of the edges of the lid, or recesses on walls of cavities of the container and tab projections on the lid.

In accordance with some embodiments of the invention, the TEE Probe container, comprises: a container configured for receiving a TEE Probe, the container comprising a first container surface and an opposing second container surface, wherein the container comprises: one or more depressions positioned on the first surface; and a guard positioned on an extending along the perimeter of the first container surface; a planar lid configured to removably enclose the first container surface of the container.

In some embodiments, or in combination with any of the previous embodiments, the one or more depressions comprise: a first depression positioned of the first container surface configured for receiving the TEE probe; a second depression positioned of the first container surface configured for receiving a sponge; a third depression positioned of the first container surface configured for receiving a mouthpiece; and a fourth depression positioned on the first container surface for receiving a flexible probe cover/sleeve that is configured to cover at least a portion of the TEE probe. For instance, the flexible probe cover/sleeve is configured to cover the TEE probe shaft. Alternatively, in some embodiments, or in combination with any of the previous embodiments, the one or more depressions comprise: a first depression positioned of the first container surface configured for receiving the TEE probe; a second depression positioned of the first container surface configured for receiving a sponge; and a third depression positioned of the first container surface configured for receiving a mouthpiece.

In some embodiments, or in combination with any of the previous embodiments, the first depression, the second depression, the third depression, and the fourth depression are discrete. In alternative embodiments, or in combination with any of the previous embodiments, the first depression, the second depression and the third depression are discrete.

In some embodiments, or in combination with any of the previous embodiments, the TEE Probe Container further comprises a flexible container bag configured to encase at least a portion of the TEE probe, for example, the TEE probe shaft. Alternatively, in some embodiments, or in combination with any of the previous embodiments, the TEE Probe Container further comprises a flexible container bag configured to encase the container.

In accordance with some embodiments of the invention, a method of using a TEE Probe container configured for transporting a TEE Probe comprises: providing a TEE probe container comprising: a container configured for receiving a TEE Probe, the container comprising a first container surface and an opposing second container surface and one or more depressions positioned on the first surface; and a planar lid configured to removably enclose the first container surface of the container; enclosing the TEE probe shaft with a flexible probe cover/sleeve; and assembling the lid and the container. Alternatively, in accordance with some embodiments of the invention, a method of using a TEE Probe container configured for transporting a TEE Probe comprises: providing a TEE probe container comprising: a container configured for receiving a TEE Probe, the container comprising a first container surface and an opposing second container surface and one or more depressions positioned on the first surface; and a planar lid configured to removably enclose the first container surface of the container; enclosing the container with a flexible container bag; enclosing the lid with a flexible lid bag; and assembling the enclosed lid and the enclosed container.

In some embodiments, the TEE probe container comprises at least four depressions positioned on the container surface, which may or may not be discrete from one another. The first depression is configured for receiving the TEE probe, the second depression is configured for receiving a sponge, the third depression is configured for receiving a mouthpiece, and fourth depression is configured for receiving a flexible probe cover/sleeve that is configured to cover at least a portion of the TEE probe, for example, the TEE probe shaft. In some embodiments, the container includes a guard extending along the perimeter of the first container surface that aids in keeping fluids, such as biofluid, deposited on the first container surface within the boundaries of the container surface. Alternatively, in some embodiments, the TEE probe container comprises at least three depressions positioned on the container surface, which may or may not be discrete from one another. The first depression is configured for receiving the TEE probe, the second depression is configured for receiving a sponge, and the third depression is configured for receiving a mouthpiece. In some embodiments, the container includes a guard extending along the perimeter of the first container surface that aids in keeping fluids, such as biofluid, deposited on the first container surface within the boundaries of the container surface. The TEE probe container apparatus may include removable, flexible bags to encase the lid, and removable, flexible bags to encase the container surface.

In some embodiments, or in combination with any of the above embodiments, the method of using the TEE probe container further comprises: removing the lid and the container; applying a flexible probe cover over at least a portion of the TEE Probe; utilizing the TEE Probe in a procedure; replacing the TEE Probe in a TEE Probe depression of the one or more depressions of the container after the procedure; and reassembling the lid to the container.

In some embodiments, or in combination with any of the above embodiments, the method of using the TEE probe container further comprises: utilizing a sponge and a mouthpiece in the procedure; replacing the sponge and the mouthpiece after the procedure in a sponge depression and in a mouthpiece depression of the one or more depressions positioned on the first surface; and discarding the flexible probe cover, replacing the flexible probe cover in a flexible probe cover depression, or retaining the flexible probe cover on at least a portion of the TEE Probe.

Unless otherwise defined, all technical and scientific terms used herein have the same meaning as commonly understood by one of ordinary skill in the art to which this invention belongs. Although methods and materials similar, or equivalent to those described herein can be used in the practice or testing of the present invention, suitable methods and materials are described below. In case of conflict, the patent specification, including definitions, will control. In addition, the materials, methods, and examples are illustrative only and are not intended to be limiting.

The features, functions, and advantages that have been discussed may be achieved independently in various embodiments of the present invention or may be combined with yet other embodiments, further details of which can be seen with reference to the following description and drawings.

### BRIEF DESCRIPTION OF THE DRAWINGS

Having thus described embodiments of the invention in general terms, reference will now be made to the accompanying drawings, wherein:

FIG. 1 illustrates a perspective view of a TEE Probe container 100, in accordance with some embodiments of the invention;

FIG. 2 illustrates a perspective exploded view of a TEE Probe container 100, in accordance with some embodiments of the invention;

FIG. 3a illustrates a top view of the TEE Probe container 100 without a lid, in accordance with some embodiments of the invention;

FIG. 3b illustrates a side view of the TEE Probe container 100 of FIG. 3a;

FIG. 4 illustrates a perspective view of a TEE Probe container 200, in accordance with some embodiments of the invention;

FIG. 5 illustrates a perspective exploded view of a TEE Probe container 200, in accordance with some embodiments of the invention;

FIG. 6a illustrates a top view of the TEE Probe container 200 without a lid, in accordance with some embodiments of the invention;

FIG. 6b illustrates a side view of the TEE Probe container 200 of FIG. 3a; and

FIG. 7 illustrates a high level process flow 300 for a method of assembly and use of the TEE probe container 100 and/or 200, in accordance with some embodiments of the invention.

Some embodiments of the invention are herein described, by way of example only, with reference to the accompanying drawings. With specific reference to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. The description taken with the drawings makes apparent to those skilled in the art how the various forms of the invention may be embodied in practice.

### DETAILED DESCRIPTION OF EMBODIMENTS OF THE INVENTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to elements throughout. Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

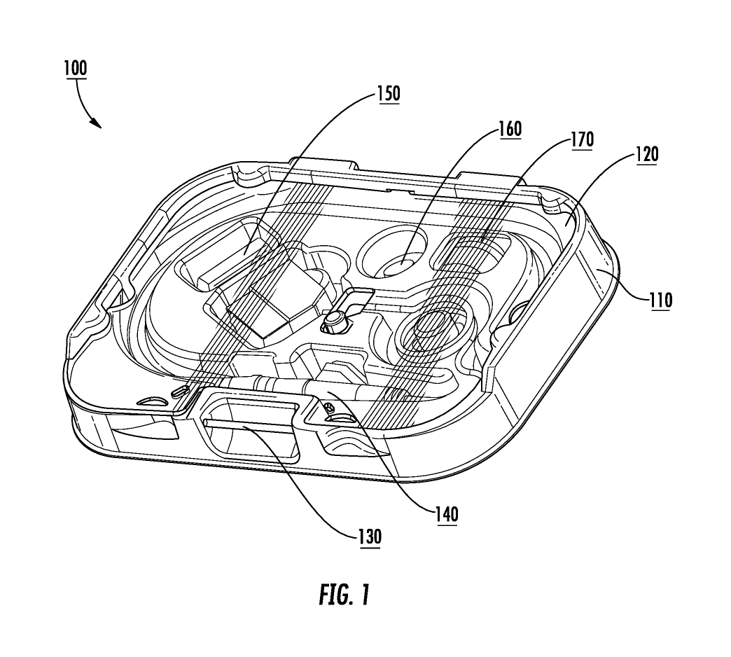

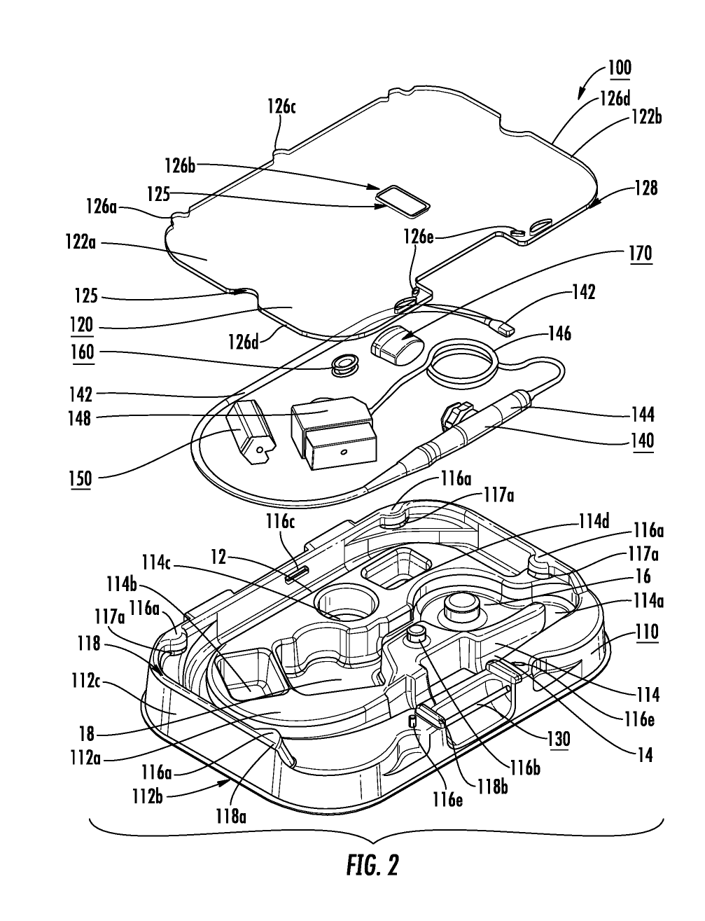

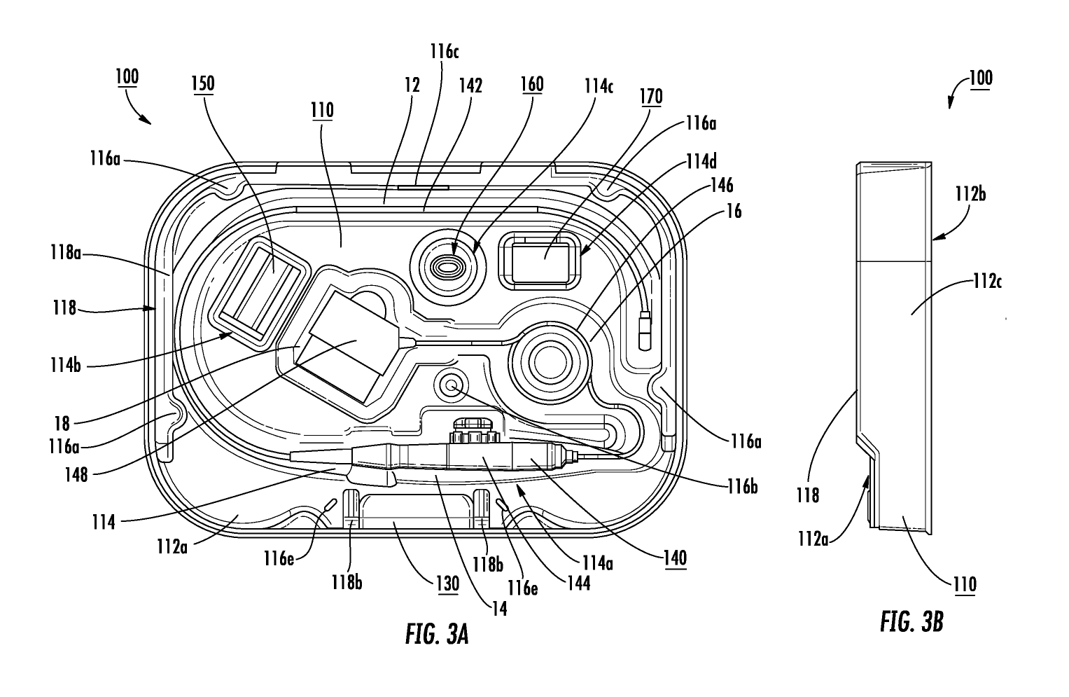

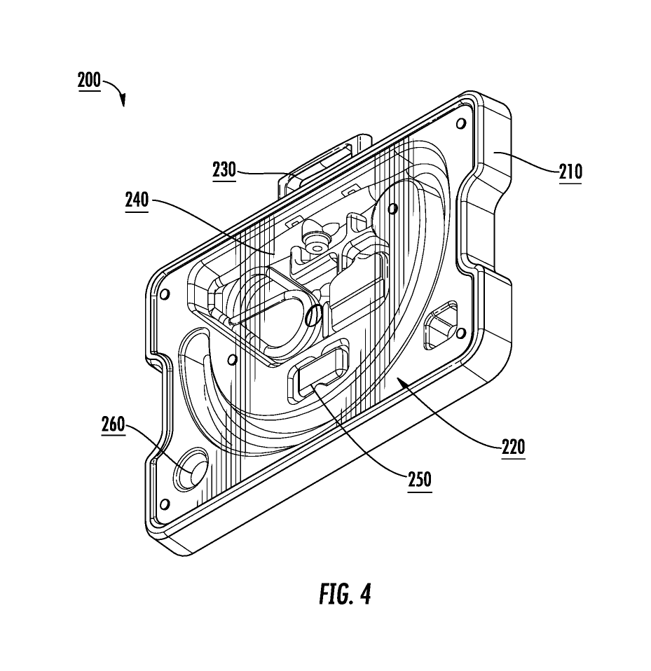

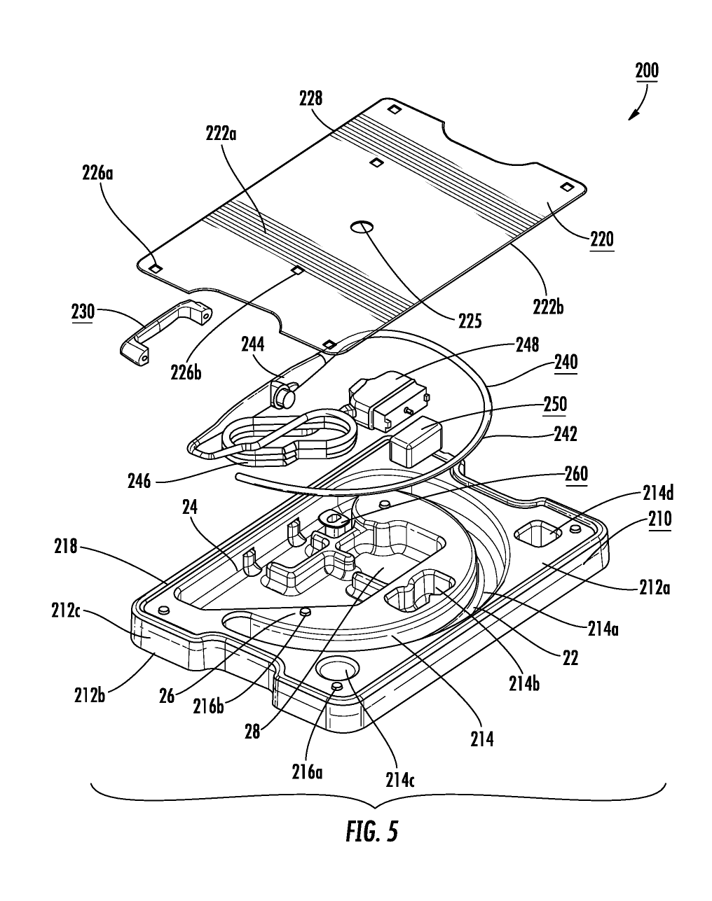

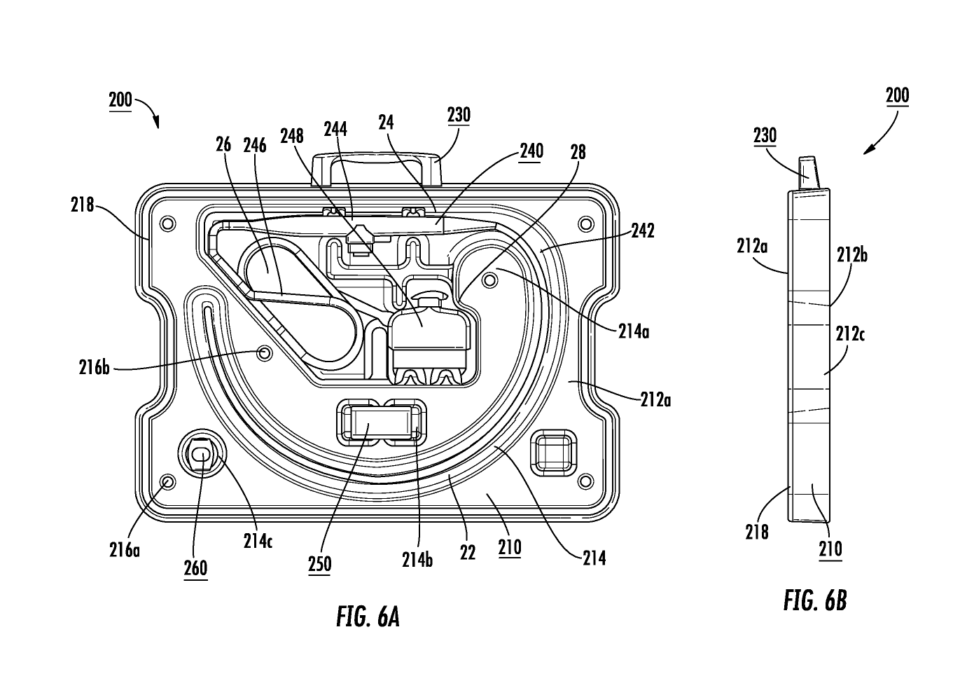

FIG. 1 illustrates a perspective view of a Transesophageal Echocardiogram (TEE) Probe container 100 or a container assembly 100, in accordance with some embodiments of the invention. The TEE Probe Container 100 typically comprises an assembly configured to receive and transport a TEE Probe 140 and/or one or more associated components (a sponge 150 and/or a mouthpiece 160 and/or a flexible probe cover 170 or flexible probe sleeve 170 configured to cover at least a portion of the TEE Probe 140). In some instances, the flexible probe sleeve 170 is configured to cover a TEE Probe shaft of the TEE Probe 140. In some instances, the TEE Probe shaft is a portion/component of the TEE probe 140 that is configured to be inserted into a patient (for example, probe portion 142 that is inserted into the patient's esophagus through the patient's mouth illustrated in FIGS. 2 and 3a, or probe portion 242 illustrated in FIGS. 5 and 6a). That said, in other instances, the TEE probe shaft may refer to a portion/component of the TEE probe 140 that is not configured to be inserted into a patient (for example, a probe handle (144, 244), a connector cable (146, 246) and/or an electric connection device (148, 248) illustrated in FIGS. 2, 5) respectively). As illustrated in FIG. 1, the TEE Probe Container 100 typically comprises a container 110 and a lid 120. In some instances, a handle 130 is also provided. In some instances, the handle 130 is integral with the container 110 or lid 120, while in other instances, the handle 130 is a separate component that may be secured to the container 110 and/or lid 120. In some embodiments, for example, in the embodiments of the container 100 illustrated in FIGS. 1-3, or in the alternative embodiments of the container 200 illustrated in FIGS. 4-6b, the TEE Probe Container assembly (100, 200) may further comprise a container disposable bag or flexible container bag (not illustrated) that is configured to fit over the container 110 before assembly of the lid 120, and a lid disposable bag or flexible lid bag (not illustrated) that is configured to encase the lid 120 before assembly. The container disposable bag is configured to conform to the contours of the container 110. Typically, the container disposable bag and the lid disposable bag each comprise an opening configured to receive the container 110 and the lid 120, respectively. Although termed as a TEE Probe container 100, it is understood that in some embodiments, the TEE probe container 100 and the container 110 in particular are configured to receive and transport surgical/medical probes, endoscopes, and/or other medical instruments/devices that may be susceptible/prone to contamination with bioburden, pathogen, foreign materials, or other biological/chemical contamination during use and/or medical instruments/devices that may require disinfection/sterilization after use.

The components of the TEE Probe Container 100 will now be described in detail with respect to FIG. 2 which illustrates a perspective exploded view of a TEE Probe container 100. The container 110 typically comprises a first container surface 112a and an opposing second container surface 112b. The container 110 further comprises lateral sides 112c terminating the first container surface 112a and the second container surface 112b. In some embodiments, the first container surface 112a, the second container surface 112b, and/or the lateral sides 112c are three-dimensional contoured surfaces, while in other embodiments they are flat surfaces, while in yet other embodiments they may be tiered surfaces. Furthermore, the first container surface 112a, the second container surface 112b, and/or the lateral sides 112c may be shaped like a polygon, an ellipse, or any other suitable curvilinear shape with sharp and/or curved corners. Typically, the lateral sides 112c are oriented at an angle or oriented transversely with respect to the first container surface 112a, and/or the second container surface 112b.

In some instances, the container 110 further comprises a guard 118 positioned at and extending along (or proximate to) the perimeter of the first container surface 112a. The guard 118 is typically configured such that inner lateral sides of the guard 118 surround or substantially surround a first volume that is bounded on one side by the first container surface 112a. In some embodiments, the guard 118 is oriented parallel to and/or positioned coincident with ends of the lateral sides 112c. In other embodiments the guard 118 is oriented transversely with respect to the lateral sides 112c. Moreover, the guard 118 may be coincident and/or contiguous with the ends of the lateral sides 112c or the guard may be offset from the ends of the lateral sides 112c. The guard 118 may comprise a suitable cross-section that may either be constant or variable throughout the extent of the guard 118.

Moreover, the guard 118 typically extends along at least a portion of the perimeter of the first container surface 112a, either in a continuous or discontinuous manner. For example, in some embodiments, the guard 118 (for example, the guard 118 illustrated in FIG. 2) extends along a portion of the perimeter of the first container surface 112a, in a discontinuous manner with a first guard portion 118a extending substantially along three of the lateral sides 112c and other discrete second guard portions 118b provided proximate the handle 130. Alternatively, in some embodiments, the guard 218 (for example, the guard 218 of TEE Probe Container 200, illustrated in FIGS. 5-6a) extends along the entirety of the perimeter of the first container surface 212a, in a continuous manner such that such that inner lateral sides of the guard 218 surround a first volume that is bounded on one side by the first container surface 212a (illustrated in FIGS. 5-6a).

Typically, the container 110 further comprises one or more container depressions or cavities 114 on the first container surface 112a, positioned adjacent to the first volume, for receiving the TEE Probe 140 and/or the other associated devices/components (150, 160, 170). The one or more container depressions 114 are suitably shaped and dimensioned to at least partially enclose the TEE Probe 140 and/or the other components (150, 160, 170). In some embodiments, the one or more container depressions 114 extend away from the first container surface 112a and in a direction towards the second container surface 112b. As such, the one or more container depressions 114 each define a constant or variable depression depth D, that may each be greater than, equal to or lesser than a thickness of the lateral sides 112c.

Typically, each container depression of the one or more container depressions 114 is sized, dimensioned and configured to receive at least one of the TEE Probe 140 or the other associated components (150, 160, 170). In this regard, the container depression 114 typically comprises a TEE probe depression 114a that is configured to receive the TEE probe 140. In addition, in some embodiments, the container depression 114 includes a sponge depression 114b configured for receiving the sponge 150, a mouthpiece depressions 114c configured for receiving the mouthpiece 160, a probe cover depression 114d configured for receiving a flexible bag that is configured to at least partially cover/enclose the TEE probe 140 (for example, cover at least a portion of the TEE probe shaft), and/or other depressions or cavities. FIGS. 2 and 3 illustrate one embodiment of the container 110 comprising at least four container depressions or cavities (114a, 114b, 114c, 114d) that are configured for receiving the TEE probe 140, the sponge 150, the mouthpiece 160, and a flexible probe cover or sleeve 170 to cover the TEE probe shaft, respectively. In some embodiments, the one or more container depressions 114 may be discrete or separated from one another, while in other embodiments at least some of the one or more container depressions 114 may be contiguous. In some embodiments, for example, those illustrated with respect to FIGS. 5-6a, the container depressions 214 include at least three container depressions or cavities (214a, 214b, 214c) that are configured for receiving the TEE probe 240, the sponge 250, and the mouthpiece 260, respectively.

As discussed, in some embodiments, the one or more container depressions 114 further comprise the probe cover depression 114d or sleeve depression 114d configured for receiving the flexible probe cover or sleeve 170, the flexible probe cover 170 being configured for covering at least a portion of the TEE probe (for example the flexible cover 170 may be configured for covering the TEE probe shaft). In some instances, the flexible probe cover or sleeve 170 is a disposable cover/jacket that is configured to sheath/receive/enclose at least a portion of the TEE probe 140. In this regard, in some instances the flexible probe cover or sleeve 170 or the TEE probe cover/sleeve 170 is designed to conform to the corresponding contours of the TEE probe 140 and designed to take the shape of the portion that is envelops. For example, the flexible probe cover or sleeve 170 may be a flexible hollow elongate case or a tube like structure configured for receiving a probe portion 142 of the TEE probe 140 that is typically inserted into the patient's esophagus. As such, an unused TEE probe sleeve may be stored/placed in the probe cover depression 114d, which may then be retrieved and used to additionally cover/sheath and protect the TEE probe 140 before placing a used TEE probe 140 in the container 110 (for example, to cover the probe portion 142 to prevent the probe from contaminating its surroundings and vice versa). As another example, the TEE probe sleeve 170 may be stored/placed in the sleeve depression 114d when transporting a decontaminated TEE probe 140 for pre-patient-use (for example, to prevent contamination), and/or a contaminated TEE probe 140 for post-patient-use. In some instances, the probe cover depression 114d may be dimensioned and sized to receive a rolled, folded or otherwise collapsed/gathered TEE probe sleeve 170 or the flexible probe cover or sleeve 170, while in other instances the probe cover depression 114d may be dimensioned to receive the TEE probe sleeve 170 or flexible probe cover or sleeve 170 in its open state (e.g., in the cases in which the sleeve is hard, is flexible but generally retains its shape, or the like).

In some embodiments, the one or more container depressions 114 may comprise an auxiliary depression (not illustrated) that is configured to receive another object that could be associated with the TEE probe, such as an ultrasound gel (not illustrated) and/or a bite-holder (not illustrated), or the depression may be provided to facilitate or may be a result of the manufacturing process used to fabricate the container 110. It should be understood that the TEE probe depression 114a, the sponge depression 114b, the mouthpiece depression 114c, the probe cover depression 114d and/or the auxiliary depression, also referred to as the first, second, third, fourth and/or fifth depressions respectively, may be positioned in any suitable location in the container 110 and in any suitable arrangement. As such, in some instances, the one or more depressions 114 may comprise a plurality of depressions (114a, 114b, 114c and/or 114d), as illustrated by FIG. 2. Although illustrated as being adjacent one another along the first container surface 112a, it is also understood that a plurality of depressions (114a, 114b, 114c and/or 114d) may be stacked in a plane transverse to the first surface 112a, with one depression leading to or atop another partially or entirely.

In some embodiments, each of the container depressions 114 may comprise a plurality of portions that are each sized and dimensioned to accommodate and/or position in a predetermined manner, at least a portion of a corresponding device. For instance, the TEE Probe 140 may be comprised of separate portions/components, including a probe 142, a probe handle 144, a connector cable 146 and an electric connection device 148 or connection device 148. Here, the TEE Probe depression 114a may comprise four distinct/contiguous portions including a probe depression portion 12, a probe handle depression portion 14, a connector cable depression portion 16 and a connection device depression portion 18 configured for receiving/accommodating the corresponding portions of the TEE probe 140. In some instances, a pair of the plurality of portions may be at least partially separated by walls, protrusions and the like. For instance, FIG. 2 illustrates a pair of walls substantially separating the connector cable depression portion 16 and the connection device depression portion 18. The walls may be configured such that the at least a portion of the TEE probe 140 may be accommodated and held in a predetermined manner in a recess between the walls, for example, to prevent sliding of the TEE probe 140 when the TEE probe 140 is placed in the container depression 114a. The walls may also be configured to prevent fluid flow from one portion to another. For instance, the walls may prevent contaminants and other fluids on portions of the TEE probe 140 from flowing into the connection device depression portion 18, to prevent contamination of the connection device 148 and/or prevent damage to electric components of the connection device 148 from fluid contact, when the TEE probe 140 is placed in the container depression 114a. For instance, FIG. 3 illustrates the TEE probe 140 being inserted or positioned in the designated depression 114a with the electric connection device 148 being positioned in the connection device depression portion 18. In this instance, the electric connection device 148 and/or the connection device depression portion 18 are substantially separated from the connector cable depression portion 16 by the walls in the depression 114a.

Furthermore, the container 110 may comprise one or more container fastening members, suitably located on the container surfaces/sides. In some embodiments, the container 110 comprises one or more distal container fastening members 116a positioned on the first surface 112a towards the lateral sides 112c, and/or one or more proximal container fastening members 116b positioned towards a central location of the first surface 112a. In this regard, in some embodiments, the container 110 comprises four corner container fastening members 116a (distal) on the first surface 112a proximate each of the four corners and a container fastening member 116b (proximal) positioned slightly offset from a central location of the first surface 112a, as illustrated by FIGS. 2 and 3. In this regard, the arrangement of the proximal and distal container fastening members and the offset is configured such that the container 110 is non-symmetrical about the first surface 112a. In other embodiments, for example the embodiments illustrated with respect to FIGS. 4-6a, the container 210 comprises four corner container fastening members 216a (distal) on the first surface 212a proximate each of the four corners and two staggered container fastening members 216b (proximal) positioned in a staggered manner around a central location of the first surface 212a. In some instances, these non-symmetrical or staggered arrangements of the container fastening members (116a-b, 216a-b), described above, are configured such that the lid (120, 220) is irreversible, in that the lid securely couples to the container (110, 210) in only one position, as will be described in detail below.

In other embodiments, one or more container fastening members may be positioned symmetrically on the first container surface 112a, for example by positioning the one or more proximal container fastening members 116b equidistantly from opposing lateral sides instead of a staggered arrangement. In some embodiments, the one or more container fastening members, for example the four corner container fastening members 116a and/or the offset container fastening member 116b may be protrusions that extend away from the first surface 112a, as illustrated by FIG. 2. Similarly, as depicted in FIG. 5, the four corner container fastening members 216a and/or the two staggered container fastening members 216b may be protrusions that extend away from the first surface 212a. Although in other embodiments, the container fastening members may be depressions or other suitable shapes. In addition, one or more of the container fastening members may comprise additional coupling elements for securely coupling the lid 120. For example, as illustrated by FIG. 2, each of the distal container fastening members 116a may comprise a groove coupling element 117a, that is configured to receive a corresponding edge portion 126d of the lid 120 or a distal lid fastening member 126a comprising a tab projection protruding from an edge of the lid 120, when the container fastening members are coupled with the lid fastening members, i.e., when the lid 120 is affixed to the container 110. That said, in alternative embodiments, the lid 120 may comprise lid grooves (for example, at lateral edges/faces between lid surfaces 122a and 122b) that are configured to mate with tab projections provided on the container 110 (for example, along inner lateral sides of the guard 118, along inner lateral walls of the cavities 114, or the like).

Moreover, as illustrated in FIG. 2, the container fastening members may further comprise a lateral container fastening member 116c provided on a wall of one of the cavities 114. For example, as illustrated, the lateral container fastening member 116c may comprise a recess configured for receiving a corresponding projection coupling element 126c (e.g., lid tab or tab projection) of the lid 120, when the lid 120 is affixed to the container 110. In addition, in some embodiments, the container fastening members may further comprise one or more auxiliary container fastening members 116e in the form of protrusions or projections extending from the surface 112a. This auxiliary container fastening members 116e are configured to be received by corresponding apertures 126e of the lid 120, when the lid 120 is coupled to the container 110. That said, in alternative embodiments not illustrated herein, the container 110 may comprise the apertures or depressions that are configured to receive or mate with the auxiliary lid fastening members in the form of protrusions/projections provided on the lid 120, for example on a second lid surface 122b of the lid 120.

That said, it is understood that the container fastening members 116 may comprise any combination of depressions, protrusions, grooves, or any suitable contour configured for engaging the corresponding lid fastening members 126.

The TEE Probe container assembly 110 may further comprise the handle 130 that is configured to be attached to a lateral side 112c of the container 110.

Typically, the TEE Probe container assembly 100 further comprises a lid 120. Typically, the lid 120 is unattached or separate from the container 110, although it is contemplated that in alternate embodiments the lid 120 may be at least partially attached to the container 110, or may be attached using a hinge arrangement. In some embodiments the lid 120 is a planar member with a first lid surface 122a and an opposing second lid surface 122b defining a perimeter 128. In some embodiments, the perimeter 128 of the lid 120 comprises dimensions and contours that are substantially similar to the first container surface 112a substantially within the guard 118 and/or lateral sides 112c of the container 110. The lid 120 is shaped and dimensioned such that the second lid surface 122b is configured to contact the first container surface 112a, on assembly (for example, with portions of the disposable bags located between).

Furthermore, the lid 120 may comprise one or more lid fastening members including distal lid fastening members 126a and/or one or more proximal lid fastening members 126b, whose contour and position typically complements the corresponding one or more distal container fastening members 116a and/or the one or more proximal container fastening members 116b, respectively. For instance, as illustrated by FIG. 2, the lid fastening members of the lid 120 comprise distal lid fastening members 126a, a proximal lid fastening member 126b, a projection coupling element 126c, edge portions 126d, and an aperture 126e located on the lid 120 such that the lid fastening members are proximate to/engage with corresponding container fastening members (116a, 116b, 116c, 117a and 116e, respectively) when the container 110 and the lid 120 are assembled. In this regard, the lid fastening members and corresponding container fastening members may form fastening member pairs on assembly. As discussed, the distal lid fastening members 126a and the edge portions 126d may be received within the groove coupling elements 117a of the corresponding container fastening members 116a. The proximal lid fastening member 126b and the apertures 126e may be through holes (or recesses in alternative embodiments), which are sized and dimensioned to receive through them, the protrusions of the proximal container fastening member 116b and the auxiliary container fastening members 116e, respectively, on assembly. Moreover, the projection coupling element 126c of the lid 120 may be received within the recess of the lateral container fastening member 116c. That said, it is understood that the lid fastening members 126 may comprise any combinations of depressions, protrusions or any suitable contour configured for engaging the corresponding container fastening members 116. In some instances, the lid 120 further comprises one or more apertures or recesses 125 for facilitating removal of the lid 120 from the container 110 during disassembly.

In some embodiments, the centrally offset proximal container fastening member 116b of the container 110, and the corresponding proximal lid fastening member 126b of the lid 120 are positioned in a non-symmetrical manner such that the lid 120 is irreversible. In this regard, the irreversible lid 120 may be assembled with the container 110 only when the second lid surface 122b faces the first container surface 112a, because rotating or flipping the lid 120 over would prevent the proximal lid fastening member 126b from receiving the protrusion of the proximal container fastening member 116b.

However, in other embodiments, for example in the instances where the fastening members are positioned symmetrically, the lid 120 is reversible (not illustrated). In some embodiments, the container fastening members (116a, 116b, 116c, 116e) and the corresponding lid fastening members ((126a, 126d), 126b, 126d, 126e) might be arranged in a manner conducive to a securing a reversible lid 120. In some embodiments, the lid 120 may be rotated, flipped, or both, and still retain its ability to fasten to the container 110 surface. The invention encompasses any number of possible complimentary pair designs for the fasteners (116a, 116b, 116c, 116e) and accompanying fastener attachments on the lid. For assembly, the lid 120 is releasably attached to the container 110 by positioning the second lid surface 122b proximate the first container surface 112a such that the container fastening members (116a, 116b, 116c, 116e) engage the corresponding lid fastening members ((126a, 126d), 126b, 126d, 126e), respectively. In some embodiments, the lid 110 may be flexible enough to allow the aforementioned engagement of the fastening members.

During use, storage and transport of the TEE Probe 140 and the other components like the sponge 150, mouthpiece 160, and the flexible probe cover/sleeve 170 are first placed into the container 110. The TEE Probe 140, the sponge 150, the mouthpiece 160, and the flexible probe cover/sleeve 170 are placed in the respective depressions (114a, 114b, 114c, 114d) of the container 110. Here, in some instances, the flexible probe cover/sleeve 170 may be slid/affixed around the TEE probe 140, for example, the TEE probe shaft, prior to placing the TEE probe 140 in the container. The lid 120 is then assembled/affixed to the container, such that the TEE Probe 140 and the associated components (150, 160, 170) are completely enclosed by the first container surface 112a and the second lid surface 122b. The container fastening members (116a, 116b, 116c, 116e) engage the corresponding lid fastening members ((126a, 126d), 126b, 126d, 126e), for example with a secure fit (for example, a snap fit, an interference fit, a clearance fit, and/or the like). The guard 118 is typically configured such that, once assembled, the guard 118 prevents flow of fluids or contaminations from the TEE probe and other components outside the assembly 100. As such, contaminated TEE probe and components may be securely transported in this manner. The TEE Probe container assembly 100 may then be disassembled and the contaminated TEE probes and devices may be retrieved for disinfection/sterilization. The disposable cover/sleeve may then be removed and discarded. Therefore, the container 110 and the lid 120 may be reused during the transport of contaminated component and disinfected component cycles.

During a procedure involving the use of the TEE Probe, the container itself can further be viewed by one of ordinary skill in the art as a procedural kit in addition to simply a container. In some instances, a procedure involving the use of the TEE Probe container might require placement of a biteblock or mouthpiece 160 that is used during the procedure to prevent an unconscious or semi-conscious patient from biting the TEE Probe, specifically the probe portion 142 that is inserted into the patient's esophagus through the patient's mouth. In some instances, the mouthpiece 160 may be removed and placed in the TEE Probe container, and depression cavity 114c allows for the safe and sanitary placement of this device, as well as subsequent transport post-procedure. Additionally, the sanitary sponge 150 may be used in some instances to ensure that the procedural work space is clear of any fluids that may encumber the correct placement of the probe 140 and the mouthpiece 160, as well as to facilitate the correct placement of the mouthpiece, optimize the comfort of the patient and ensure that there are no unsanitary fluids on or near the procedural work space. Sponge depression 114b is present in some embodiments to provide a place for the used sponge within the container that is discrete from the other probe components. Since the sponge depression is discrete from the probe depression, the probe may be placed back in the container 110 during the procedure with little risk of contaminating other components contained within the TEE Probe container apparatus. Sanitary use and transportation of the device before, during, and after the procedure may be crucially dependent on the use of disposable bags in some embodiments. The TEE probe sleeve 170 or the flexible probe cover/sleeve 170 is typically designed to conform to the TEE Probe 140 such that any fluid or biofluid retained on the probe 140 might be kept from leaving the confines of the container apparatus.

FIG. 4 illustrates a perspective view of a Transesophageal Echocardiogram (TEE) Probe container 200, in accordance with some embodiments of the invention. The structure and functions of the container 200 and its components are substantially similar to that of the container 100 described with respect to FIGS. 1-3 previously. For example, the TEE Probe Container 200 typically comprises an assembly configured to receive and transport a TEE Probe 240 and/or one or more associated components (a sponge 250 and/or a mouthpiece 260). As illustrated in FIG. 1, the TEE Probe Container 100 typically comprises a container 210 and a lid 220. In some instances, a handle 230 is also provided. In some embodiments, the TEE Probe Container assembly 200 may further comprise a container disposable bag or flexible container bag (not illustrated) that is configured to fit over the container 210 before assembly of the lid 220, and a lid disposable bag or flexible lid bag (not illustrated) that is configured to encase the lid 220 before assembly. The container disposable bag is configured to conform to the contours of the container 210. Typically, the container disposable bag and the lid disposable bag each comprise an opening configured to receive the container 210 and the lid 220, respectively. Although termed as a TEE Probe container 100, it is understood that in some embodiments, the TEE probe container 100 and the container 210 in particular are configured to receive and transport surgical/medical probes, endoscopes, and/or other medical instruments/devices that may be susceptible/prone to contamination with bioburden, pathogen, foreign materials, or other biological/chemical contamination during use and/or medical instruments/devices that may require disinfection/sterilization after use.

As alluded to previously, in some instances, the components of the TEE Probe Container 200 are substantially similar to those described with respect to TEE Probe container 100. As discussed, the container 210 of the TEE Probe Container 200 typically comprises a first container surface 212a, a second container surface 212b, and/or lateral sides 212c. As discussed previously, in some instances, the container 210 further comprises a guard 218 positioned at and extending along (or proximate to) the perimeter of the first container surface 212a. However, the container 210 may further comprise one or more container depressions or cavities 214 similar to those described previously on the first container surface 212a, for receiving the TEE Probe 240 and/or the other associated devices/components (250, 260). Typically, as discussed previously, each container depression of the one or more container depressions 214 is sized, dimensioned and configured to receive at least one of the TEE Probe 240 or the other associated components (250, 260). In this regard, the container depression 214 typically comprises a TEE probe depression 214a that is configured to receive the TEE probe 240. In addition, in some embodiments, the container depression 214 includes a sponge depression 214b configured for receiving the sponge 250, a mouthpiece depressions 214c configured for receiving the mouthpiece 260 and/or other depressions or cavities. FIGS. 5 and 6 illustrate one embodiment of the container 210 comprising at least three container depressions or cavities (214a, 214b, 214c) that are configured for receiving the TEE probe 240, the sponge 250, and the mouthpiece 260, respectively. In some embodiments, the one or more container depressions 214 may be discrete or separated from one another, while in other embodiments at least some of the one or more container depressions 214 may be contiguous.

As discussed, the TEE Probe 240 may be comprised of separate portions/components, including a probe 242, a probe handle 244, a connector cable 246 and an electric connection device 248 or connection device 248. Here, the TEE Probe depression 214a may comprise four distinct/contiguous portions including a probe depression portion 22, a probe handle depression portion 24, a connector cable depression portion 26 and a connection device depression portion 28 configured for receiving/accommodating the corresponding portions of the TEE probe 240. In some instances, a pair of the plurality of portions may be at least partially separated by walls, protrusions and the like. For instance, FIGS. 4-6a illustrate a pair of walls substantially separating the connector cable depression portion 26 and the connection device depression portion 28. The walls may be configured such that the at least a portion of the TEE probe 240 may be accommodated and held in a predetermined manner in a recess between the walls, for example, to prevent sliding of the TEE probe 240 when the TEE probe 240 is placed in the container depression 214a. The walls may also be configured to prevent fluid flow from one portion to another. For instance, the walls may prevent contaminants and other fluids on portions of the TEE probe 240 from flowing into the connection device depression portion 28, to prevent contamination of the connection device 248 and/or prevent damage to electric components of the connection device 248 from fluid contact, when the TEE probe 240 is placed in the container depression 214a.

Moreover, the TEE Probe container assembly 200 may further comprise a lid 220, the lid 220 being a planar member with a first lid surface 222a and an opposing second lid surface 222b defining a perimeter 228. The perimeter 228 of the lid 220 comprises dimensions and contours that are substantially similar to the first container surface 212a within the guard 218 and/or lateral sides 212c of the container 210. The lid 220 is shaped and dimensioned such that the second lid surface 222b is configured to contact the first container surface 212a, on assembly (for example, with portions of the disposable bags located between). Moreover, as illustrated in FIGS. 5-6a, the lid 220 may comprise four corner lid fastening members 226a (distal) and two staggered lid fastening members 226b (proximal) located on the lid 220 such that the one or more lid fastening members are proximate to/engage with corresponding container fastening members when the container 210 and the lid 220 are assembled. In this regard, the lid fastening members and corresponding container fastening members may form fastening member pairs on assembly. The corner lid fastening members 226a and the staggered lid fastening members 226b may be through holes/apertures, which are sized and dimensioned to receive through them, the protrusions of the corner container fastening members 216a and the staggered container fastening members 216b respectively, on assembly. In some instances, the lid 220 further comprises an aperture/recess or finger hole 225 for facilitating removal of the lid 220 from the container 210 during disassembly. In some embodiments, the staggered container fastening members 216b and the staggered lid fastening members 226b pairs are positioned in a staggered manner such that the lid 220 is irreversible. In this regard, the irreversible lid 220 may be assembled with the container 210 only when the second lid surface 222b faces the first container surface 212a, because rotating or flipping the lid 220 over would prevent the staggered container fastening member 216b and the lid fastening member 226b pairs from engaging.

During use, storage and transport of the TEE Probe 240 and the other components like the sponge 250 and mouthpiece 260, the container disposable bag is first placed over the container 210 and the lid disposable bag is placed over the lid 220. The TEE Probe 240, the sponge 250, and the mouthpiece 260 are placed in the respective depressions (214a, 214b, 214c) of the container 210 with the container disposable bag separating the components and the container surfaces. Here, in some instances, the TEE probe sleeve may be slid/affixed around the TEE probe 240 prior to placing the probe 240 in the container. The lid 220 enclosed by the lid disposable bag is then assembled/affixed to the container, such that the TEE Probe 240 and the associated components (250, 260) are completely enclosed by the bags, the first container surface 212a, and the second lid surface 222b. The container fastening members (216a, 216b) engage the lid fastening members (226a, 226b), for example with a snap fit that also secures the container disposable bag and the lid disposable bag. As such, the container disposable bag is positioned between the first container surface 212a and the TEE Probe, and the lid disposable bag is positioned between the second lid surface 222b) and the TEE Probe. The guard 218 is typically configured such that, once assembled, the guard 218 prevents flow of fluids or contaminations from the TEE probe and other components outside the assembly 200. As such, contaminated TEE probe and components may be securely transported in this manner. The TEE Probe container assembly 200 may then be disassembled and the contaminated TEE probes and devices may be retrieved for disinfection/sterilization. The disposable bags may then be removed and discarded. The container 210 and the lid 220 may be enclosed in new disposable bags and the sterilized/disinfected components may be placed in the container 210 as described above. Therefore, the container 210 and the lid 220 may be reused during the transport of contaminated component and disinfected component cycles.

During a procedure involving the use of the TEE Probe, the container itself can further be viewed by one of ordinary skill in the art as a procedural kit in addition to simply a container. In some instances, a procedure involving the use of the TEE Probe container might require placement of a biteblock or mouthpiece 260 that is used during the procedure to prevent an unconscious or semi-conscious patient from biting the TEE Probe, specifically the probe portion 242 that is inserted into the patients esophagus through the patients mouth. In some instances, the mouthpiece 260 may be removed and placed in the TEE Probe container, and depression cavity 214c allows for the safe and sanitary placement of this device, as well as subsequent transport post-procedure. Additionally, the sanitary sponge 250 may be used in some instances to ensure that the procedural work space is clear of any fluids that may encumber the correct placement of the probe 240 and the mouthpiece 260, as well as to facilitate the correct placement of the mouthpiece, optimize the comfort of the patient and ensure that there are no unsanitary fluids on or near the procedural work space. Sponge depression 214b is present in some embodiments to provide a place for the used sponge within the container that is discrete from the other probe components. Since the sponge depression is discrete from the probe depression, the probe may be placed back in the container 210 during the procedure with little risk of contaminating other components contained within the TEE Probe container apparatus. Sanitary use and transportation of the device before, during, and after the procedure may be crucially dependent on the use of disposable bags in some embodiments. The disposable bags (and/or the TEE probe sleeve) are typically designed to conform to the TEE Probe container 210 and the lid 220 such that any fluid or biofluid retained on the probe or other components, such as the sponge 250 or mouthpiece 260 might be kept from substantially mixing together or leaving the confines of the container apparatus.

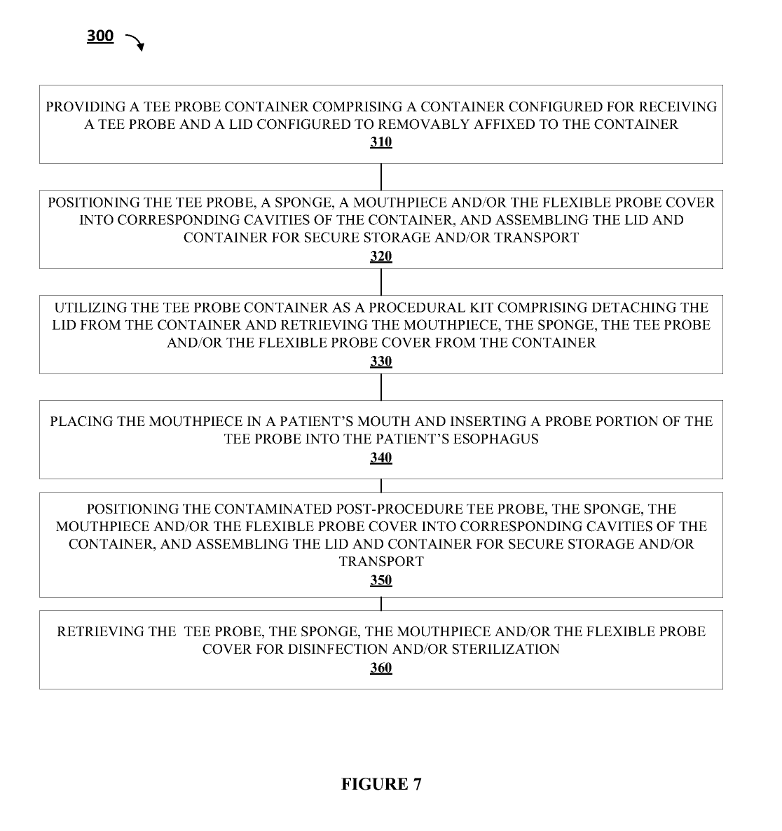

FIG. 7 illustrates a high level process flow 300 for a method of assembly and use of the TEE probe container 100 and/or 200 described previously, in accordance with some embodiments of the invention. As indicated by Block 310, a tee probe container (100, 200) comprising a container (110, 210) configured for receiving a tee probe (140, 240) and a lid configured (120, 220) to removably affixed to the container are provided, as described previously. During use, storage and/or transport of the TEE Probe (140, 240) and the other components like the sponge (150, 250), mouthpiece (160, 260), and/or the flexible probe cover/sleeve 170 are first placed into the container (110, 210), as indicated by Block 320. The TEE Probe 140, the sponge 150, the mouthpiece 160, and the flexible probe cover/sleeve 170 are placed in the respective cavities/depressions (114a, 114b, 114c, 114d) of the container 110. Here, in some instances, the flexible probe cover/sleeve 170 may be slid/affixed around the TEE probe 140, for example, the TEE probe shaft, prior to placing the TEE probe 140 in the container, or alternatively the flexible probe cover 170 is placed in the cavity 114d separate from the TEE probe 140. Subsequently, the lid (120, 220) is then assembled/affixed to the container (110, 210), such that the TEE Probe (140, 240) and the associated components ((150, 160, 170), (250, 260)) are completely enclosed by the first container surface (112a, 212a) and the second lid surface (122b, 222b). The container fastening members (for example, members 116a, 116b, 116c, 116e) engage the corresponding lid fastening members (for example, members (126a, 126d), 126b, 126d, 126e), for example with a secure fit (for example, a snap fit, an interference fit, a clearance fit, and/or the like). Alternatively, in some instances, the container disposable bag is first placed over the container (110, 210) and the lid disposable bag is placed over the lid (120, 220), prior to placing the TEE Probe (140, 240) and the other components like the sponge (150, 250) and mouthpiece (160, 260) in their respective depressions. As such, contaminated TEE probe and components may be securely transported in this manner.

Next, as illustrated by Block 330, during a procedure involving the use of the TEE Probe (140, 240), the container (100, 200) functions as a procedural kit. Typically, prior to the procedure, the lid (120, 220) is detached from the container (110, 210) and the components contained within, such as the mouthpiece, the sponge, the TEE probe and/or the flexible probe cover are retrieved from the container. The flexible probe cover may be retrieved separately from the TEE probe, in the instances where they are stored separately in the container, and the flexible probe cover may subsequently be placed over at least a portion of the TEE probe, for example, the TEE probe shaft. Alternatively, the TEE probe encased with the flexible probe cover or the TEE probe nor encases with the flexible robe cover is retrieved.

Specifically, as illustrated by Block 340, the biteblock or mouthpiece (160, 260) that configured to prevent an unconscious or semi-conscious patient from biting the TEE Probe (140, 240) during the procedure may be retrieved and appropriately positioned in the mouth of the patient. Next, the probe portion (142, 242) of the TEE probe (140, 240) may be inserted into the patient's esophagus through the patient's mouth. Here, in some instances, the probe portion may be at least partially enclosed in the flexible prove cover prior to insertion into the patient's esophagus, while in other instances the TEE probe may be inserted without the flexible probe cover. Additionally, the sanitary sponge (150, 250) may be used in some instances to ensure that the procedural work space is clear of any fluids that may encumber the correct placement of the probe (140, 240) and the mouthpiece (160, 260), as well as to facilitate the correct placement of the mouthpiece, optimize the comfort of the patient and ensure that there are no unsanitary fluids on or near the procedural work space. The procedure utilizing the TEE probe is then carried out.

During or after completion of the procedure, as indicated by Block 350, the used/contaminated mouthpiece (160, 260) may be removed and placed in the TEE Probe container (110, 210). Here, the cavity (114c, 214c) allows for the safe and sanitary placement of this device, as well as subsequent transport post-procedure. Similarly, the contaminated sponge (150, 250) may be placed in the corresponding cavity (114b, 214b). Since the mouthpiece and sponge cavities are discrete from the TEE probe cavity, the probe (140, 240) may be placed back in the container (110, 210) during and/or after the procedure with little risk of contaminating other components contained within the TEE Probe container apparatus. In some instances, the TEE Probe 140 encased in the flexible probe cover 170 is placed back into the container such that any fluid or biofluid retained on the probe 140 might be kept from leaving the confines of the container apparatus. Alternatively, the flexible probe cover 170 may then be removed and stored separately (for example, in the cavity 114d) or the flexible probe cover 170 may be discarded. The lid may then be assembled on the container for secure storage and/or transportation of the contaminated components. The guard (118, 218) is typically configured such that, once assembled, the guard (118, 218) prevents flow of fluids or contaminations from the TEE probe and other components outside the assembly (100, 200). In the embodiments where the lid and container disposable bags are employed, the lid (120, 220) enclosed by the lid disposable bag is assembled/affixed to the container (110, 210), such that the TEE Probe (140, 240) and the associated components ((150, 250), (160, 260), and/or 170) are completely enclosed by the bags, the first container surface (112a, 212a), and the second lid surface (122b, 222b). As such, the container disposable bag is positioned between the first container surface (112a, 212a) and the TEE Probe (140, 240), and the lid disposable bag is positioned between the second lid surface (122b, 222b)) and the TEE Probe.

Subsequently, as indicated by Block 360, the TEE Probe container assembly (100, 200) may then be disassembled and the contaminated TEE probes (140, 240) and devices ((150, 250), (160, 260), 170) may be retrieved for disinfection/sterilization. In some instances, the flexible prove cover 170 is removed (for example, from the probe or the container cavity) and discarded. Moreover, the lid and container bags may be disposed and/or replaced with new bags, in the embodiments employing the bags. Therefore, the container (110, 210) and the lid (120, 220) may be reused during the transport of contaminated component and disinfected component cycles, or the container (110, 210) and the lid (120, 220) may be disposable.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the Plain-English equivalents of the respective terms “comprising” and “wherein.” Moreover, in the following claims, the terms “first,” “second,” and “third,” etc. are used merely as labels, and are not intended to impose numerical requirements on their objects.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

While certain exemplary embodiments have been described and shown in the accompanying drawings, it is to be understood that such embodiments are merely illustrative of, and not restrictive on, the broad invention, and that this invention need not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A TEE Probe container, comprising:
a container configured for receiving a TEE Probe, the container comprising a first container surface and an opposing second container surface;
a lid configured to removably enclose the first container surface of the container;
wherein the container comprises:
a first depression positioned on the first container surface configured for receiving the TEE probe;
a second depression positioned on the first container surface configured for receiving a sponge;
a third depression positioned on the first container surface configured for receiving a mouthpiece; and
a fourth depression positioned on the first container surface configured for receiving a flexible probe cover.

2. The TEE probe container of claim 1, wherein the container further comprises a guard positioned on and extending along a perimeter of the first container surface.

3. The TEE probe container of claim 1, wherein the first depression, the second depression, the third depression and the fourth depression are discrete.

4. The TEE probe container of claim 1, wherein the flexible probe cover is configured to encase at least a portion of the TEE Probe.

5. The TEE probe container of claim 1, wherein the flexible probe cover is configured to encase the pre-patient use TEE Probe.

6. The TEE probe container of claim 1, wherein the flexible probe cover is configured to encase the post-patient use TEE Probe.

7. The TEE probe container of claim 1, wherein the lid is operatively coupled to the container through one or more fastening members, wherein the fastening members comprises mating protrusions and apertures, groove elements and tab projections, groove elements and a portions of the edges of the lid, or recesses on walls of cavities of the container and tab projections on the lid.

8. A TEE Probe container, comprising:
a container configured for receiving a TEE Probe, the container comprising a first container surface and an opposing second container surface, wherein the container comprises:
one or more depressions positioned on the first surface; and
a guard positioned on an extending along a perimeter of the first container surface;
a lid configured to removably enclose the first container surface of the container.

9. The TEE probe container of claim 8, wherein the one or more depressions comprise:
a first depression positioned on the first container surface configured for receiving the TEE probe;
a second depression positioned on the first container surface configured for receiving a sponge;
a third depression positioned on the first container surface configured for receiving a mouthpiece; and
a fourth depression positioned on the first container surface configured for receiving a flexible probe cover.

10. The TEE probe container of claim 8, wherein the first depression, the second depression, the third depression and the fourth depression are discrete.

11. The TEE probe container of claim 8, wherein the flexible probe cover is configured to encase at least a portion of the TEE Probe.

12. The TEE probe container of claim 8, wherein the flexible probe cover is configured to encase at least a portion of the pre-patient use TEE Probe.

13. The TEE probe container of claim 8, wherein the flexible probe cover is configured to encase the post-patient use TEE Probe.

14. The TEE probe container of claim 8, wherein the TEE Probe Container further comprises a flexible container bag configured to encase the container.

15. The TEE probe container of claim 8, wherein the TEE Probe Container further comprises a flexible lid bag configured to encase the lid.

16. The TEE probe container of claim 8, wherein the lid is operatively coupled to the container through one or more fastening members, wherein the fastening members comprises mating protrusions and apertures, groove elements and tab projections, groove elements and a portions of the edges of the lid, or recesses on walls of cavities of the container and tab projections on the lid.

17. A method of using a TEE Probe container configured for transporting a TEE Probe, comprising:
providing a TEE probe container comprising:
a container configured for receiving a TEE Probe, the container comprising a first container surface and an opposing second container surface and one or more depressions positioned on the first surface; and
a planar lid configured to removably enclose the first container surface of the container;
enclosing the TEE Probe with a flexible probe cover; and
assembling the lid and the container.

18. The method of claim 17, further comprising:
removing the lid and the container;
applying a flexible probe cover over at least a portion of the TEE Probe;
utilizing the TEE Probe in a procedure;
replacing the TEE Probe in a TEE Probe depression of the one or more depressions of the container after the procedure; and
reassembling the lid to the container.

19. The method of claim 18, further comprising:
utilizing a sponge and a mouthpiece in the procedure;
replacing the sponge and the mouthpiece after the procedure in a sponge depression and in a mouthpiece depression of the one or more depressions positioned on the first surface; and
discarding the flexible probe cover, replacing the flexible probe cover in a flexible probe cover depression, or retaining the flexible probe cover on at least a portion of the TEE Probe.
